(19) United States
(12) Patent Application Publication
(10) Pub. No.: US 2021/0282931 A1
(43) Pub. Date: Sep. 16, 2021

(54) IMPLANT FOR USE IN A WEAR COUPLE INCLUDING A SPHERICAL WEAR PARTNER

(71) Applicant: CeramTec GmbH, Plochingen (DE)

(72) Inventor: Mateusz Maria JUSZCZYK, Velden (DE)

(21) Appl. No.: 17/053,276

(22) PCT Filed: May 6, 2019
(86) PCT No.: PCT/EP2019/061518

§ 371 Date: Nov. 5, 2020

(30) Foreign Application Priority Data
May 7, 2018 (EP) 18171105.2

(51) Int. Cl. A61F 2/30

(52) U.S. Cl. CPC A61F 2/30; A61F 2002/302; A61F 2002/30655; A61F 2/32; A61F 2310/00179; A61F 2002/30784; A61F 2310/00592; A61F 2002/30242

(57) ABSTRACT

The invention describes an implant for wear couples in endoprosthetics, the implant having an outer side, with an outer face, and an inner side, and a hemispherical wear region for accommodating a spherical wear partner being formed on the inner side. The aim of the invention is to reduce the height of the implant as much as possible and to ensure that, e.g., the pelvic bone does not have to be milled down as much. According to the invention, the implant is therefore designed in the form of a ring or annular structure and the outer face permits direct implantation in the body. In order to reduce friction between the spherical wear partner and the implant to a minimum, the implant has a specially designed inner geometry.

The invention relates to an implant for tribological pairing in endoprosthesis, the implant comprising an outside and an inside or inner surface, and a specially designed sliding region for receiving a spherical sliding partner being formed on the inner surface. This is preferably a ceramic implant.

Hitherto, in endoprosthesis implants consisting of a metal shell and a ceramic half-shell insert were used. The metal shell is also designed as a half-shell and receives the ceramic insert. The sliding partner, the prosthesis head, is spherical and is received by the ceramic insert. Ceramic inserts for tribological pairing in hip replacement are hemispherical and cover approximately 50% of the prosthesis head. The central point of the sliding surface rests on the plane of the end face of the insert, or slightly thereabove or therebelow. The outside of the insert is divided into a plurality of regions. The region of the outside, on the equator, comprises a clamping surface which may be conical or cylindrical. By means of said clamping surface an operative connection to a shell, usually a metal shell, is established. The insert is inserted into the shell. This is carried out either in a manner already premounted following production, or only during implantation.

A further region of the outside, the rear face of the insert, which extends from the equator to the pole, is not in contact with the metal shell but, for reasons of stability, must have a minimum wall thickness.

In the case of this pairing, the load transfer between the femoral head and insert or acetabulum in the sliding surface takes place in a punctual or circumferential manner, since there is a positive clearance between the sphere diameter of the prosthesis head and the indentation diameter of the insert. In this case, the load is transferred to the insert from the femoral head in an axiparallel manner.

DE 10 2016 222 616 A1 discloses an implant comprising a ceramic annular insert which is introduced into a metal shell and comprises, on the inside, a hemispherical sliding surface for receiving the spherical sliding partner. The overall depth for the metal shell comprising the annular insert is reduced, such that, in comparison with a conventional implant consisting of a metal shell and ceramic half-shell insert a less deep recess is required in the pelvic bone. Furthermore, there are no punctual loads, but rather strip-like loads having reduced maximum values, similar to the physiological load bearing capacity.

Proceeding therefrom, the object was that of further reducing the friction between the spherical sliding partner and the implant. Furthermore, the object consisted in developing an implant for endoprosthesis that is as cost-effective and simple as possible, which can be implanted in the bone in a simple manner.

This object is achieved according to the invention by a half-shell, preferably annular, implant according to the features of claim 1. Preferred embodiments are specified in the dependent claims. Embodiments can be combined with one another as desired.

According to the invention, the implant is designed such that it is anchored directly in the bone, without a metal shell. The design of the outer surface on the outside firstly allows for the use of cement-free direct implantation, but also for implantation by means of medical adhesive and/or cement. The implant is preferably formed so as to be ceramic at least in part, preferably entirely ceramic.

The implant is used, according to the invention, for receiving a sphere of a prosthesis head, i.e. a spherical sliding partner. The sphere of the prosthetic head and the implant form a tribological pairing. The implant is retained in a stationary manner in the pelvic bone. The sphere of the prosthesis head should be able to rotate in the implant. In this case, it should be possible to prevent the sphere of the prosthesis from springing out.

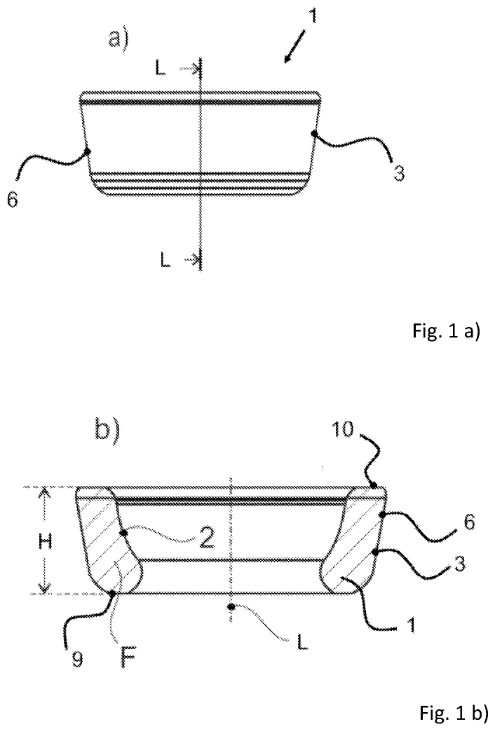

In the present case, an implant, preferably an annular implant (ring), is to be understood as a member that is formed of a cross-sectional surface F (see FIG. 1b) which rotates about an axis of rotation L (see FIG. 1b). It comprises a concave inner surface and an outside. The shape of the outside can be formed in a manner deviating from the shape of the inside.

The implant comprises a first region, comprising an end face and an infeed zone which ensures the introduction of a sphere of a prosthesis head of the spherical sliding partner into the implant, and a second region which limits the reception of the sphere. In one embodiment, the implant comprises a half shell, the second region of which is closed. In a further embodiment, the implant corresponds to a ring, the second region of which, comprising a base surface and a discharge zone, is open. The circular opening of the first region, of the receiving region, has a diameter that is greater than the diameter of the opening of the second region. In this case, the circular opening of the second region of the annular implant is smaller than the diameter of the spherical sliding partner to be inserted, in order to prevent the spherical sliding partner, referred to in the following as KG, from slipping out.

The connection between the inner surface and the outside forms a transition and is preferably established by means of radii. The rounded transitions prevent sharp edges and corners, as a result of which the stability of the implant is improved. In addition, this facilitates the handling. These radii are preferably of a size of 0.5-2 mm. In the first region of the implant, the first transition of the inner surface to the outside comprises an end face.

In the case of the half-shell embodiment, the outside is closed. The surface development of the outside can correspond to a closed circle. The second region of the implant which is arranged opposite the first region is closed and comprises a closed base surface. The outside of said closed base surface is part of the outside of the implant. The maximum spacing between the first region, in which the end face is arranged, and the base surface, corresponds to the height H of the half shell. The inner surface of the closed base surface is part of the inner surface of the implant. The inner surface of the implant comprises a sliding region which is adjoined by the inner surface of the closed base surface. A half-shell embodiment of an implant comprises a first opening, and an infeed zone for introducing a sphere. The geometry of the inner surface of the closed base surface can correspond to a dome, a half-sphere, or a shape similar to a half-sphere.

The annular embodiment of the implant comprises a second opening opposite the first region. The diameter of said second opening is smaller than the diameter of the first opening of the first region. As a result, the introduction of the KG into the implant is made possible and is limited. The surface development of the outside of the annular insert corresponds to a ring. As a result of the opening, which is arranged opposite the first region of the implant, this comprises a second transition between the inner surface and the outside. This is located in the second region and limits the height of the implant. The transition between the inner surface and the outside comprises a base surface. The maximum spacing between the end face and the second transition or the base surface corresponds to the height H of the annular implant.

The rounded first transition of the inner surface of the implant, as far as the start of the sliding region, is referred to as the infeed zone, the rounded second transition of the inner surface of the implant, as far as the start of the sliding region, is referred to as the discharge zone.

The inner surface is designed so as to be rotationally symmetrical, at least in part. The outside and/or outer surface of the implant, preferably of the annular implant, can deviate from the rotational symmetry in regions. The height H (see FIG. 1b) of the implant is understood to be the extension thereof along the axis of rotation L. In the annular embodiment, the height H is substantially smaller compared with the half-shell embodiment. The outside of the implant corresponds to the side which faces the bone in which the implant is intended to be implanted. The outer surface is a region of the outside and is used for fastening the implant in the bone. The size of the outer surface can correspond to the size of the outside. It can also be designed so as to be smaller. The outer surface can assume various shapes, or be divided into a plurality of regions or individual surfaces which are interconnected or delimited from one another. The design of the individual surfaces can be the same or different.

The implant according to the invention is designed as a half shell or ring such that it interacts with spheres according to the prior art, the functionality being ensured. The implant has a wall thickness of at least 3 mm, in order to ensure the stability. The maximum wall thickness of the implant depends on the sinter properties of the material used, and is in the region of 15 mm, preferably a maximum of 15 mm. The height H of the annular implant is preferably 5-20 mm. Depending on the situation during use, an implant having the appropriate geometric dimensions is used.

The inner surface of the implant comprises a sliding region on which the KG is intended to rotate. The sliding region of the implant is concave and corresponds to a portion of a surface of a rotation member.

The rotation member is a spindle torus which is described by a circle 108 that rotates about an axis of rotation. The spacing A of the axis of rotation from the center point M′/M″ of the circle is smaller than the radius r of the circle describing the torus. The center point straight lines L′ and L″ are located in parallel with the axis of rotation L. The torus describes, in the inside, a spindle 105 having a center point M which is located in the center of the straight line that describes the maximum longitudinal extension of the spindle 105 and rests on the axis of rotation. The points of intersection of the outer surface of the spindle 105 with the axis L are designated E and E′. In this case, the surface is the outer surface 106 of the spindle 105 thus described.

The portion 107 that describes the sliding region of the implant corresponds to the region between the two normal planes S and S′ which intersect the longitudinal axis L, which corresponds to the axis of rotation of the spindle torus, at points S1 and S2. Both points of intersection are located between E′ and M, i.e. in one half of the spindle 105, in the longitudinal extension. S1 can correspond to the center point M of the spindle 105. S2 is located between S1 and E′ or corresponds to E′.

Therefore, in the simplest embodiment thereof the implant has an inside geometry which corresponds to a portion of the spindle of a spindle torus, the region of the inner surface that is located between the end face and the base surface being concave, and it being possible for the geometry of the outside to deviate from the rotational symmetry. The sliding region of the inner surface is therefore not hemispherical, i.e. does not correspond to a portion of a sphere. The sliding region corresponds to the portion 107 of the outer surface 106 of a spindle 105. The portion 107 is located in one half of the spindle 105, along the longitudinal axis thereof, and does not exceed the center point M of the spindle on the longitudinal axis L of the spindle 105. In the direction of the end face, the sliding region of the implant has a maximum diameter D1 at the first opening thereof. At the second opening, the sliding region has a minimum diameter D2. The diameter D1 of the implant is larger than the diameter D2. The diameters of the spindle 105 between D1 and D2 reduce in size in the direction D2.

The inside geometry according to the invention ensures the movability of the KG, i.e. a sphere or a sphere portion of the prosthesis head. The diameter D1 of the first opening of the implant is larger than the outside diameter of the KG which is introduced into the implant. The diameter D2 is smaller than the outside diameter of the KG. The smallest diameter of the infeed zone is preferably larger than D1.

In one embodiment, S2 corresponds to the point E′. If S2 corresponds to E′, the implant is a half shell. In this embodiment of the half-shell implant, the diameter D2=0, i.e. no second opening is present.

In a further embodiment, the implant is a half shell and S2 is located above E′ on the axis L. In this embodiment, the inner surface is flattened in the region E′. The height H of the implant reduces as a result. In this embodiment of the half-shell implant, the geometry of the inner surface of the closed base surface deviates from the geometry of the spindle. In this case, care is preferably taken that the flattened inner surface of the closed base surface should be designed such that it does not influence the geometry of the contact line, and sufficient space is provided for the KG, in order not to create any point-based friction. This is then a hemispherical or preferably further flattened inner surface of the closed base surface.

In a preferred embodiment, S2 is located above E′ on the axis L, and the discharge zone adjoins the sliding region at D2. The implant is then a ring. In the annular embodiment, D2 is smaller than the radius of the KG to be inserted, in order to prevent falling out.

The KG thus rotates in the non-hemispherical sliding region of the inner surface, the sliding region corresponding to a portion 107 of half a spindle of a spindle torus in the longitudinal extension.

The height HG of the sliding region corresponds to at least 20% and at most 80% of the diameter of the KG to be inserted, and preferably 50-95% of the height H of the implant.

The height of the sliding region corresponds to the extension in the longitudinal direction, i.e. along the axis of rotation L. The height HG preferably corresponds to at least 25%, particularly preferably at least 30%, and preferably at most 70%, particularly preferably at most 60%, of the diameter of the KG to be inserted. For an annular implant, the height HG is in particular at most 50% of the diameter of the KG.

In one embodiment, the KG has a diameter of 5-70 mm, preferably 6-64 mm. KG for human artificial joints have a diameter of 20-70 mm, preferably 22-64 mm, and for animal artificial joints KG having a diameter of 5-20 mm, preferably 6-19 mm, are used. As a result, in this embodiment an implant in which a KG having a diameter of 5 mm is intended to be inserted has a sliding region having a height of at least 1 mm and at most 4 mm.

Furthermore, in one embodiment the height H_{G} of the sliding region (2) corresponds to at least 20%, preferably at least 35%, particularly preferably at least 50%, and at most 95%, of the height H of the half-shell or annular implant. The discharge and infeed zone of an annular implant are not part of the sliding region. The infeed zone of the half-shell implant, and the inner surface of the closed base surface having a possible flattening, are not part of the sliding region. In the mounted state, the KG preferably does not touch the infeed and inner surface of the closed base surface of the half-shell implant.

With respect to the geometry of the spindle, the following conditions preferably apply:
A is the distance between L and L″ or the horizontal distance from the center point to the axis of rotation.
r is the radius of the circle describing the spindle torus.
r_{P} is the radius of the spherical sliding partner, i.e. the radius of the prosthesis head.
C is the clearance and complies with formula I

C=(r−r_{P})*2 (formula I).

In one embodiment, the clearance corresponds to the sum of the maximum deviations, specified in production, of the extensions of the prosthesis head (of the radius r_{P}) and of an implant that has a hemispherical sliding region and is suitable for the prosthesis head. In a particular embodiment, C>10 μm, preferably >25 μm, particularly preferably 50 μm, and <500 μm, preferably <350 μm, and particularly preferably 280 μm.
The radius r of the circle 108 describing the spindle torus is larger than the radius r_{P} of the KG.

KG is in contact with the sliding region and slides thereon; KG is preferably in linear contact with the sliding region of the implant.

The contact line corresponds to a circular line in the sliding region, i.e. in the portion 107 on the outer surface 106 of the spindle 105 of the spindle torus. This line corresponds to the line of intersection of a sectional plane 111 through the spindle 105 in the region between S and S′. In the case of a firmly specified radius r_{P} of the prosthesis head and a firmly specified clearance, the diameter of the annular contact or the annular contact line can be influenced by changing the distance A. As a result, the angle α which between the longitudinal axis L of the spindle and the straight line, which connects the center point of the spherical sliding partner to a point 110 on the contact line, can be influenced. If a increases, the contact line is oriented in the direction of the infeed zone of the implant. If a decreases, the contact line is oriented in the direction of the base surface or discharge zone. Point contact would exist in a hemispherical implant if α=0. Since the implant according to the invention in the half-shell shape has a spindle shape, the sphere cannot touch the point of intersection E′.

In one embodiment of the annular implant the contact line is located in the lower half of the height H of the implant, i.e. in the half of the implant facing the second region. Viewed from the base surface or the discharge zone, the contact line is thus in the range of 0-50% of the height. As a result, the dislocation, i.e. the prosthesis head springing out of the implant, is counteracted. The contact line is preferably between 10-40% and particularly preferably between 20-30% of the width of the implant, viewed from the base surface. Said contact line, arranged at a distance from the base surface, also allows for the formation of lubrication film, e.g. consisting of synovial fluid, which assists the sliding of the sphere in the implant.

In the annular embodiment thereof the implant according to the invention has a substantially smaller height, and thus a substantially smaller installation depth, compared with a conventional half-shell implant. The recess in the bone for the implants can therefore be smaller. This allows for the use of an artificial implant in very small or thin bones, in particular hip bones, as frequently occur in teenagers or children or animals. An implant according to the invention having a reduced height makes it possible for the depth required for inserting the implant to be reduced to a minimum.

Preferably, the concave sliding region extends over 80%, particularly preferably over 95%, most particularly preferably over the entire inner surface of the implant, as a result of which a large portion or the entire inner surface is available for the tribological pairing.

The center point of the sliding region is preferably arranged on the plane of the end face, or slightly thereabove or therebelow, in the range of 0 to 2 mm.

In a further embodiment of the implant according to the invention, the implant, preferably also the sliding region, is formed so as to be extended on the portion of the implant, along the longitudinal axis. This means that the height H of the implant, and preferably also the height HG of the sliding region, change over the periphery of the circle. In this case, the implant, preferably also the sliding region, is formed so as to be elevated/extended either in the direction of the prosthesis head, beyond the end face of the implant, and/or, in the case of an annular insert, beyond the base surface of the implant.

This enlargement of the implant, preferably of the sliding region, is referred to as cranial enlargement, and comprises just a part, a portion of the peripheral surface of the implant. As a result, the tendency for dislocation is reduced. In this case, the center of rotation is preferably located on or below the end face.

A region or portion of the implant that is arranged in the region of the infeed zone is referred to as the cranial elevation. The height H of the implant expands as a result. In one embodiment, this elevation also lengthens the sliding region.

A region or a portion of the implant that is arranged in the region of the discharge zone is referred to as a cranial lengthening. In one embodiment, this lengthening elevation also enlarges the sliding region.

In one embodiment the cranial enlargement of the implant is formed by a balcony-like protrusion or a shaped projection, the inside of which is a continuation of the receiving space described by the circle lines or of the inner surface of the implant. In this case, the protrusion preferably makes up′ of the surface described by said circle lines, on which surface the cranial elevation and/or lengthening is located.

In another embodiment of an implant according to the invention, the end face is not arranged in a plane. By means of the continuous slope of the end face and/or of the base surface (in the case of an annular implant) of the implant, the cranial enlargement is achieved. Proceeding from a position on the end face (or base surface), said end face (or base surface) rises continuously until it has reached its highest point after 180 degrees. Proceeding from this highest point, the end face (or base surface) then drops again continuously as far as the starting point thereof. As a result, the end face or base surface is arranged at a shallow angle with respect to the axis of rotation R. The shallow angle of the tilted end face thus arranged is 95 to 105 degrees, preferably 97 to 101 degrees, particularly preferably 99.5 degrees. In this case, the center point of the sliding region is on or below the end face. The continuous ascent in the end or base surface can also be in a range that is less than 180 degrees. The same applies for the descent. The ascent and decent are preferably of the same length, but can also be of different lengths.

Owing to the cranial elevation, the maximum height H′ of the implant deviates, in the region of the cranial enlargement, from the height H of the implant without the cranial enlargement. The following applies for the height of the implant: H′=H+x+y. In one embodiment, the cranial enlargement also leads to an elevation of the sliding region; this applies analogously for the height of the sliding region: H_{G}′=H_{G}+x_{G}=y_{G}.

The maximum extension of the cranial lengthening is denoted by x. This is the distance between the sectional plane S′ and the point Y′. The distance x thus describes the height difference of the points X′ and Y′ along the axis of rotation L.

The maximum extension of the sliding region of the cranial lengthening is denoted x_{G}.

The maximum extension of the cranial elevation is denoted y. This is the distance between the sectional plane S and the point Y. The distance y thus describes the height difference of the points X and Y along the axis of rotation L.

The maximum extension of the sliding region of the cranial elevation is denoted y_{G} and describes the height difference of points X_{G} and Y_{G}.

If x=y=0, no cranial enlargement exists.

If x>0 and y=0, a cranial lengthening exists. In this case, in addition, in a preferred embodiment x_{G}>0.

If x=0 and y=0 a cranial elevation exists. In this case, in a preferred embodiment the following additionally applies: y_{G}>0.

In a further embodiment, the following applies: x>0 and y>0, where x=I≠y and x_{G}=I≠y_{G}≥0.

The distances x and y are directly proportional, depending on the diameter of the sphere, to be used, of the prosthesis head, and the sum of x+y is preferably 2-20 mm, particularly preferably 3-15 mm.

In one embodiment, the cranial elevation follows the geometry of the spindle. That is to say that the portion of the torus that forms the lengthened region of the implant, optionally the lengthened sliding region, of the cranial elevation, is a continuation of the geometry of the spindle.

In another embodiment, an implant having a cranial enlargement no longer has any rotational symmetry along the axis of rotation L, in the region of the cranial enlargement. In one embodiment, the radii of the implant, optionally of the sliding region, of the cranial enlargement are not oriented to the geometry of a spindle.

The value of the radius that defines the sliding region can then deviate from the value of the radius that defines the elevation. The value of said radius (of the elevation) can preferably be smaller than or equal to D1/2.

In this case, the cranial elevation must always fulfil the condition that the spherical sliding partner can still be inserted, i.e. the opening has a diameter that is larger than the diameter of the KG. The sectional plane through the spindle, which is located between a point X, which lies on the plane S and the outer surface of the spindle, and a further point Y, which lies opposite X and represents the maximum of the cranial elevation, must be of a diameter that corresponds at least to that of the region of the spherical sliding partner that is to be inserted. In this case, X lies on the opposing side from Y, i.e. a straight line K from X to Y intersects L. The preferred maximum cranial elevation of the implant results from a straight line K between X and Y, if this also intersects the center point of the spindle. This preferably also applies for a cranial elevation of the sliding region.

Particularly preferably, Y lies on the outer surface of the spindle. In this case, the inner surface further corresponds to a portion of a spindle, the part of the implant that completely surrounds the sliding partner in a circular manner corresponds to the portion of half of the spindle along the longitudinal axis thereof, and this part does not exceed the center point of the longitudinal axis L of the spindle.

In one embodiment of the cranial lengthening, the radii of the implant, optionally of the sliding region, of the cranial enlargement are not oriented to the geometry of a spindle. The value of the radius that defines the sliding region can then deviate from the value of the radius that defines the lengthening. The value of said radius (the elevation) can be smaller than or equal to

$$\frac{D 2}{2} .$$

In a further embodiment, the portion of the torus that forms the lengthened sliding region is a continuation of the geometry of the spindle.

In this case, the cranial lengthening must always fulfil the condition that the spherical sliding partner still cannot fall out, i.e. the opening has a diameter that is smaller than the diameter of the KG. The sectional plane through the spindle, which is located between a point X′, which lies on the plane S′ and the outer surface of the spindle, and a further point Y′, which lies opposite X′ and represents the maximum of the cranial lengthening, must be of a diameter that is smaller than the diameter of the spherical sliding partner. In this case, X′ lies on the opposite side from Y′, i.e. a straight line K′ from X′ to Y′ intersects L. In this case, Y′ is particularly preferably also located on the outer surface of the spindle.

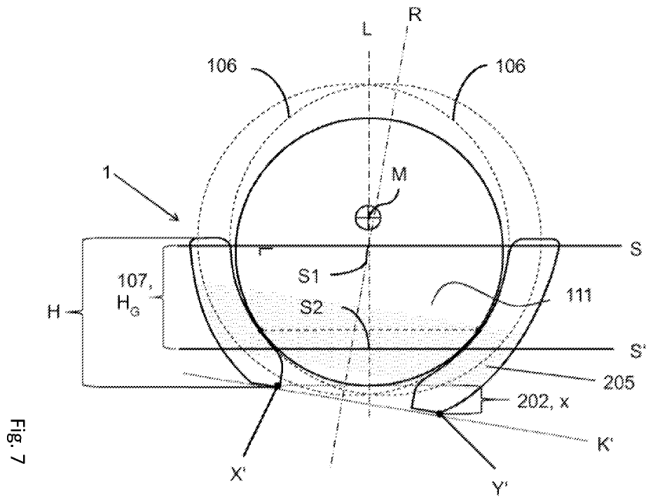

In one embodiment, the axis of rotation L does not correspond to the axis of rotation R of the conical or cylindrical outside of the insert, preferably in the case of an annular insert having a cranial lengthening. The axis of rotation R preferably intersects the axis of rotation L, particularly preferably in the region of the sliding region. The axis of rotation R is preferably arranged such that it is perpendicular to and intersects the straight line K′ which interconnects the maximum extension of the base surface of the annular insert with and without a cranial lengthening, in points X′ and Y′. If an insert of this kind is inserted into a conventional shell, the cranial lengthening appears as a cranial elevation, and the inside geometry corresponds to a tilted spindle (shown in FIG. 12).

In a preferred embodiment, the implant is intended to meet, in a high-quality manner, the mechanical, biological, and chemical requirements during the implantation and during the time in the human or animal body. The implant is therefore preferably produced from a ceramic material. For this purpose, the implant further comprises an outer surface on the outside, which outer surface is designed such that human or animal bone cells (e.g. osteoblasts) or cells which are required for formation of human or animal bone tissue (ossification) find favorable conditions. The implant is preferably designed such that it can be anchored directly in the bone, via the outer surface. For this purpose, the outer surface comprises means for anchoring the implant. Thus, direct implantation of the implant in the bone is possible. The aim of an embodiment of this kind is that of complete osseous installation in human or animal bones.

In a preferred embodiment, the outer surface of the implant is provided with a rough surface which allows for direct implantation and fixing in the bone. In this case, the rough surface assists the engraftment of the implant into the bone, and thus also takes on the task of anchoring in the bone. According to the invention, a rough surface is understood to mean an outer surface which has a three-dimensional structure or also roughness, in the form of elevations and depressions. These elevations and depressions have dimensions that do not change the original shape of an implant according to the invention. For example, this means that an implant having a circular outside that has a rough outer surface still has a circular shape. Furthermore, this also applies for all other shapes.

In one embodiment, the rough surface of the outer surface is achieved by means of a metal coating of the implant and/or by applying one or more chemical compounds which promote osseointegration. The one or more chemical compounds are preferably selected from hydroxylapatite, tricalcium phosphate, or other calcium phosphates and/or mixtures thereof. The metal coating consists primarily of the materials which are conventionally also used as metal shells for prostheses. The metal coatings are preferably titanium-containing coatings, particularly preferably titanium particles. The layer thickness of the metal and/or chemical coating is 100-400 μm, preferably 175-300 μm, and has a roughness Rₐ (average roughness) of 15-50 μm. In order to produce the implant coated in this way, in one embodiment firstly the uncoated ceramic implant is created and sintered. Subsequently, the coating is applied to the outside of the ceramic implant, at least in part, and thus forms an outer surface having a rough surface. Preferably, the metal and/or chemical coating is applied by means of thermal spraying, particularly preferably by means of plasma spraying.

In a further embodiment, the rough surface of the outer surface is achieved by means of a porous surface on the outside. In addition to the primary anchoring, the porous surface of the outer surface also allows for engraftment of the bone for increased long-term stability.

It has been found that the following properties of a porous surface have a positive influence on the ossification:
porosities between 50% and 99%, preferably between 60% and 85%
interconnectivity, i.e. the individual pores are interconnected at least in part
pore sizes between 100-1000 μm.

In one embodiment, the porous surface consists of a metal, porous layer. This metal, porous layer preferably contains titanium particles, particularly preferably it consists of titanium particles. The metal, porous layer is preferably 100-500 μm thick.

In a preferred embodiment, the porous surface is achieved by means of porous ceramic which is applied to the outside of the implant at least in part. In a particularly preferred embodiment, the chemical composition of the porous ceramic of the implant according to the invention has the same chemical composition as the remainder of the implant. As a porous surface, the porous ceramic has an arithmetic average roughness of Rₐ=10-100 μm, preferably Rₐ=20-80 μm. The porosity of the porous layer is between 50% and 99%, preferably between 60% and 85%, particularly preferably between 60 and 80%, and the pores have a pore size between 100-1000 μm.

Preferably 10%-90% of the cumulative surface portion of the outer surface of the implant has pores of a size of 100-600 μm. In a particular embodiment, an implant comprises an outer surface in which at least 80% of the pore surface is in contact with the bone, and these pores are of a size of 100-600 μm.

In general, a plurality of methods and processes for producing the porous ceramics are known. These include slurry-based methods in which ceramic porous layers are produced on parts, or completely porous parts are produced, by means of a ceramic slurry comprising organic, structure-determining porosity enhancers or chemical ingredients. The ceramic slurries are to be understood as suspensions which comprise a fluid medium, a ceramic starting powder, and optionally additional additives.

In a preferred embodiment, the porous ceramic consists of a ceramic foam made of the solid ceramic material. In one embodiment, the implant consists of 100% ceramic foam. In a preferred embodiment, the ceramic foam consists of a mixed oxide system Al₂O₃—ZrO₂, in particular ZTA ceramics (zirconia toughened alumina), or ceramic composite materials in which zirconium oxide is the volume-dominant phase, and, depending on the dominant phase, chemical stabilizers or dispersoids in the form of further metal oxides or mixed oxides also being added to said systems.

Using a ceramic foam significantly changes the behavior of the implant. Therefore, in the case of local high loads, in particular under pressure, it is possible for only a locally limited defect to result instead of catastrophic failure of the entire implant. The local defect or the local damage is revealed in the form of breaks in the pore ribs, and is limited to the region that comprises the porous foam.

The porous layer of the ceramic implant is furthermore also adhesively bondable and cementable. In this case, the porosity of the porous layer is advantageous, since the implant is infiltrated with the adhesive/bone cement (>0.5 mm deep), and, in addition to a chemical connection, is also mechanically connected, for example interlocked, therewith. As a result, connections to other materials such as non-ceramic materials such as plastics materials and metals are also possible. If, for medical reasons, the bone structure at the recessed location is not suitable for cement-free implantation, fastening by means of bone cement can thus be avoided. The decision regarding the additional use of bone cement can be made during the application. The user therefore has the option of deciding, during insertion of the implant, whether the bone structure allows for cement-free insertion or whether cemented insertion is required.

The implant comprising an outer surface made of a porous ceramic foam is produced by means of various methods, but the manufacture preferably takes place directly during the production process of the ceramic implant. The direct manufacturing processes include for example a two-stage injection molding process in which, preferably, initially the first part is cast in a die, and subsequently, in particular by means of suitable modification of the die, the further part is cast, which further part surrounds the first part and forms the structured and/or rough outer surface of the outside, at least in part. Subsequently, co-sintering of the two parts takes place, as well as final processing, which ensures that the pores on the outer surface are opened by means of, for example, the ceramic material being for example applied on the surface of the ceramic material.

One embodiment of the invention comprises a structured surface having recesses and/or projections on the outer surface. This structured surface corresponds to a macroscopic three-dimensional structure of the outer surface which is used for counteracting rotation or displacement of the implant in the bone. This means that the shape of the outside is determined by the macroscopic recesses and/or projections. The macroscopic three-dimensional structure can be formed as projections, teeth or undulations. As a result, the permanent anchoring in the bone can be ensured. The structured surface firstly ensures slip-free, stationary connection in the bone since, in a first phase of remaining in the human body the implant is held in the correct position simply by the mechanical properties thereof. By means of the recesses and/or projections the implant hooks into the bone or the implant is fixed in the position in which it was inserted.

In this case, a corrugated surface means that a 3-dimensional structure is formed on the surface by periodically recurring local minima and maxima, having the same or different distances between the minima and/or maxima, and the same or different height differences between adjacent minima and maxima.

In one embodiment, the implant comprises an outside and/or outer surface having a rough surface which is additionally structured, i.e. the rough, in a preferred embodiment porous, surface additionally has a macroscopic, three-dimensional structure.

In a preferred embodiment the outside, preferably the rough, particularly preferably the porous, surface of the outer surface is in addition activated such that it promotes cell growth by means of biological activation applied to the outer surface and/or the outside, of a layer containing amino acids, peptides and/or proteins such as RGD peptide (Arg-Gly-Asp) and/or collagen.

In a particularly preferred embodiment the ceramic implant is designed such that it comprises a macroscopically three-dimensionally structured, rough, and/or porous and activated outer surface. In one embodiment, the outside comprises differently structured and/or rough and/or porous surfaces with or without biological activation, side-by-side.

The implant is preferably fully ceramic. The implant according to the invention preferably consists of an oxidic ceramic. Oxidic ceramics are characterized in particular by a high degree of stability and good compatibility with body media. Oxidic ceramics exhibit a high degree of biocompatibility and cause virtually no allergic reactions.

According to a preferred embodiment of the invention, the implant is based on oxidic ceramic material systems, comprising:
zirconium oxide-toughened aluminum oxide (ZTA) and all ZTA systems developed on this basis.
zirconium oxide ceramic, in particular yttrium-stabilized zirconium oxide (3Y-TZP).
cer-stabilized zirconium oxide (Ce-TZP), in which the stabilization of the tetragonal phase of the zirconium oxide is achieved by cerium oxide.
all other composite materials based on zirconium oxide, it being possible for the dispersoidal compound component to be based on aluminates, and also for other stabilizers from the group of the rare earth metals to be used, such as Gd and Sm.

The implant is preferably inserted, by means of the structured and/or rough and/or porous outer surface, into the bone in a cement-free manner.

The implant is preferably used in hip, shoulder, elbow, toe joint or finger endoprosthesis in humans or animals.

Advantages are:
a lower height of the implant in the annular embodiment, and therefore a very small recess in the bone is required.
no punctual loads, but rather strip-like loads having reduced maximum values, similar to the physiological load bearing capacity.
compared with a hemispheric insert, the geometry according to the invention creates a reduced pressure on the contact point.
the implant having the geometry according to the invention can be combined, without restriction, with conventional spherical sliding partners.
the changed geometry does not have a disadvantageous effect on the production costs, since known production methods can be used.
since it is possible to omit a metal shell for anchoring and stabilization in the bone, material is saved during production, as a result of which there is a cost advantage.
during the application, depending on the situation an implant according to the invention can be inserted in a cement-free manner, or, if necessary, also using cement.

The invention describes an implant for tribological pairing in endoprosthesis, the implant comprising an outside having an outer surface and an inside having an inner surface, and a non-hemispherical sliding region for receiving a spherical sliding partner being formed on the inner surface. In order for the overall depth (height) for the implant to be as low as possible and for a less deep recess to be required for example in the pelvic bone, it is proposed, according to the invention, for the implant to preferably be formed as a ring or annularly, and for the outer surface to allow for direct implantation into the body. In order to minimize the friction between the spherical sliding partner and the implant, a specifically designed inside geometry of the implant is proposed.

In summary, the implant (1) for the tribological pairing with a spherical sliding partner (5) is designed as a half shell or in an annular manner, and comprises an inner surface which is formed as the sliding region (2) for receiving a spherical sliding partner (5). The sliding region (2) corresponds to a portion of half a spindle of a spindle torus, in the longitudinal extension. The height H_{G} of the sliding region (2) corresponds to 20-80% of the diameter of the sphere to be inserted, and preferably 50-95% of the height of the implant.

The implant (1) preferably comprises a first region for introducing the sliding partner, and a second region that limits the reception of the sliding partner. Furthermore, the implant comprises an inner surface which is designed as the sliding region (2) for receiving a spherical sliding partner (5), an outside (6) comprising an outer surface (3) having means for anchoring the implant in the bone, and an end face (9) which represents the transition from the inside to the outside in the first region, and a base surface (10) which is located opposite the end face (9) in the second region. The sliding region (2) of the implant corresponds to a portion of half a spindle of a spindle torus in the longitudinal extension.

Selected embodiments of the annular implant according to the invention are described in the figures, in which:

FIG. 1a: is a side view of an implant,

FIG. 1b: is a cross section showing the implant according to FIG. 1a in the annular embodiment,

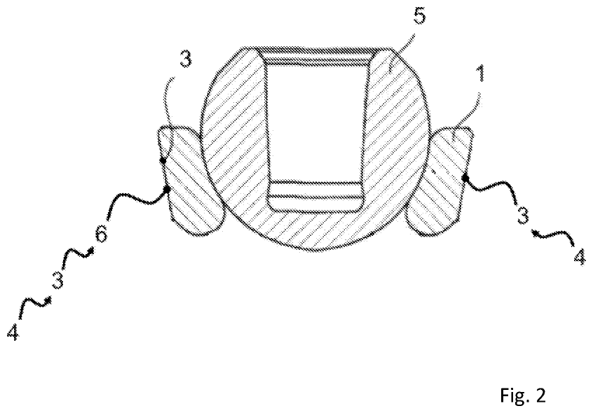

FIG. 2: shows an embodiment of an implant according to the invention comprising an inserted spherical KG,

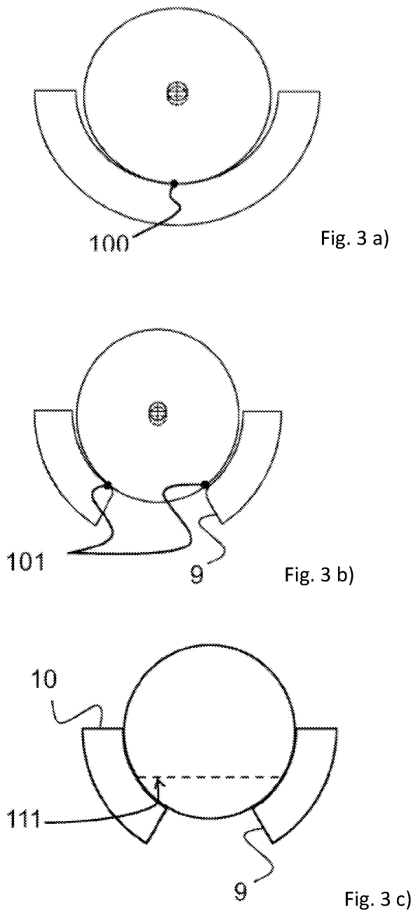

FIG. 3a-c): show the contact points of the spherical sliding partner in a conventional insert (A), a conventional annular insert (B), and the implant according to the invention (C) in the annular embodiment thereof,

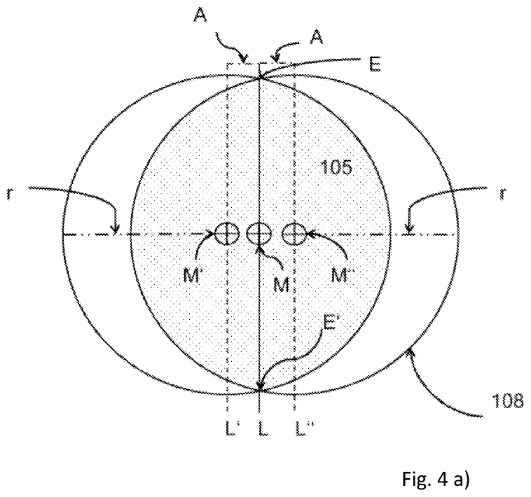

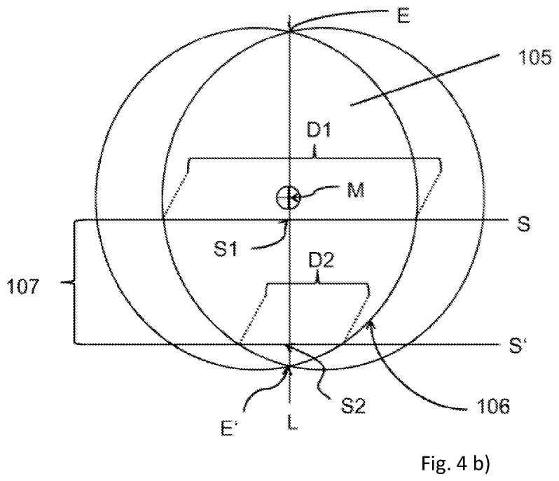

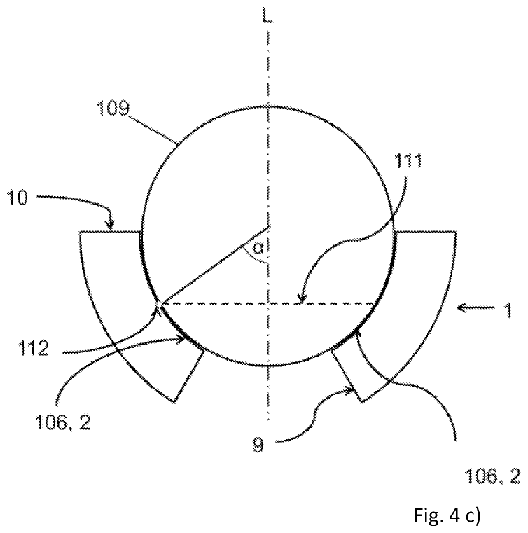

FIG. 4a-c): shows the geometry of the spindle,

FIG. 5: shows a preferred embodiment of the implant according to the invention,

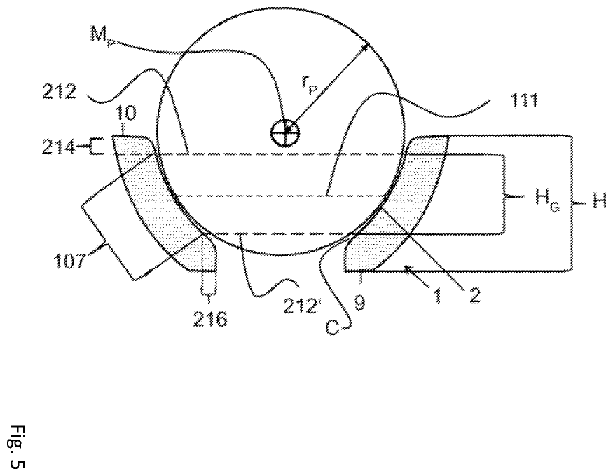

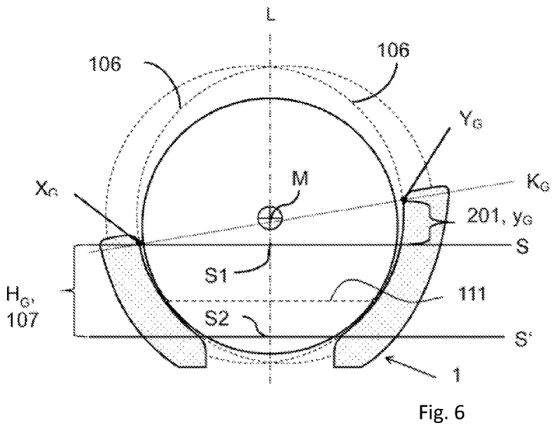

FIG. 6: shows a preferred embodiment of the geometry of the cranial elevation,

FIG. 7: shows a preferred embodiment of the geometry of the cranial lengthening.

### LIST OF REFERENCE SIGNS AND ABBREVIATIONS

1 implant
2 sliding region
3 outer surface, rough and/or structured surface
4 biological coating
5, 109 head, sphere of the prosthesis
6 outside
9 base surface
10 end face
100 contact point
101, 112 annular contact, contact line
105 spindle
106 outer surface of the spindle
107 portion of the spindle
108 circle describing the spindle torus
110 tangent point
111 sectional plane of the contact line
112 contact line
201 region of the cranial elevation of the sliding region
202 region of the cranial lengthening of the implant
205 region of the bone contact
212, 212′ circle line (orientation aid)
214 infeed zone
216 discharge zone
A distance between the axis of rotation and the center point M of the circle describing the spindle torus
C clearance
D1 maximum diameter of the sliding region, arranged in the first region
D2 minimum diameter of the sliding region, arranged in the second region
E, E′ point of intersection of the spindle outer surface with L
F cross-sectional surface
H height of the implant
H_{G} height of the sliding region
K straight line describing the cranial elevation
K_{G} straight line describing the cranial elevation of the sliding region
K′ straight line describing the cranial lengthening
K_{G} spherical sliding partner
L longitudinal axis of the spindle, axis of rotation
L′, L″ axis, in parallel with L, of the circle, describing the spindle torus, through the center point
M center point of the spindle
M′, M″ center point of the circle describing the spindle torus
M_{P} center point of the spherical sliding partner
r radius of the circle describing the spindle torus
r_{P} radius of the spherical sliding partner (K_{G}, prosthesis head)
R axis of rotation of the outer surface
S, S′ planes normal to L
S1, S2 points of intersection of the normal planes S, S′ on the longitudinal axis L
X maximum of the implant or cranial elevation in the direction of the end face
X_{G} maximum of the sliding region without a cranial elevation in the direction of the end face
X′ maximum of the implant without a cranial lengthening in the direction of the base surface
x height difference of the cranial elevation
Y maximum of the implant of the cranial elevation
Y_{G} maximum of the sliding region of the cranial elevation
Y′ maximum of the implant of the cranial lengthening
y height difference of the cranial lengthening

An annular implant 1 according to the invention is shown in FIG. 1a and FIG. 1b. FIG. 1a is a view of said implant 1 and FIG. 1b is a cross-section thereof along an axis of rotation L according to FIG. 1a. The implant 1 comprises an inner annular portion of a spindle which is also referred to as a (non-hemispheric, covalent) sliding region 2 or inner surface. In the case of hip joint prosthesis the prosthesis head 5 is articulated thereon (see FIG. 2). In a preferred embodiment an outer surface 3 having a rough and/or structured surface is arranged on the outside 6 of the implant 1, by means of which surface the implant can be anchored in the bone. The height H of the implant is shown by the dashed lines and extends from a first region, having the end face 10, over a second region, to the base surface 9. The axis of rotation is denoted by L. F denotes the cross-sectional surface of the annular implant.

FIG. 2 is a cross-sectional view of the implant 1 according to the invention. A prosthesis head 5 is inserted into the implant 1. A biological coating 4 can additionally be applied to the outside 6 or the outer surface 3.

FIG. 3a) schematically shows the most likely locations of friction of a conventional insert, FIG. 3b) shows this for a conventional annular insert, and FIG. 3c) shows this for an implant according to the invention in an annular embodiment. In the case of a known semicircular insert, the contact point 100 is positioned between the insert and K_{G}, on the base of the insert. In the case of a known annular insert, the contact between the insert and the K_{G} (FIG. 3b) takes place on a contact line 101. This is preferably a linear contact, and linear friction. This line 101 is arranged in the region close to the base surface 9. In the implant according to the invention, the correspondingly designed geometry means that the contact line is arranged on the plane 111, at a distance from the base surface 9 in the direction of the end face 10 (FIG. 3c).

FIG. 4 shows the determination of the sliding region 2 of the implant according to the invention. The spindle torus 105 in FIG. 4a) is described by a circle 108 having a radius r that has a center point M′/M″ and rotates about the axis of rotation that corresponds to the longitudinal axis L of the spindle. The axes L′ and L″ are in parallel with L and extend through M′, M″. The distance between L′/L″ and L is smaller than the radius r. The spindle intersects the longitudinal axis L in the points E and E′.

In FIG. 4b) the determination of the portion 107 is made clear. The portion 107 is located in half the spindle 105 and is formed by the planes normal to L (S and S′). Said planes intersect L in points S1 and S2 and in this case the following applies: S1=M or S1 is located between M and S2, S2=E′ or S2 is located between S1 and E′. Both the points of intersection S1, S2 are therefore located in half of the spindle and do not exceed the center thereof. The diameter D1 in the first region is larger than the diameter D2 in the second region, D1 being larger than the diameter of the K_{G} to be inserted. D2 is smaller than the diameter of the K_{G} to be inserted, as a result of which (in the case of an annular implant) the K_{G} is prevented from falling out.

FIG. 4c) schematically shows the sectional plane 111 of the contact line 112 between the K_{G} 109 and the implant 1, on the sliding region 2 thereof, according to the outer surface of the spindle 106. The contact line 112 corresponds to a sectional line 111 on the spherical sliding partner 109. Owing to the spindle shape, the region of the end face 10 is inclined towards the spherical sliding partner 109 or towards the longitudinal axis L. As a result, the diameter D1 has a smaller value compared with a diameter of a comparable hemispheric sliding region measured at the same point. As a result, the contact line 112, on which the spherical sliding partner 109 moves, is displaced towards the end face 10 of the implant and away from the base surface 9.

FIG. 5 shows the height H_{G} of the non-hemispherical sliding region 2, shown on an annular implant 1 having K_{G} inserted having a center point M_{P} and a radius r_{P}. The sliding region 2 corresponds to a portion 107 of half a spindle of a spindle torus, in the longitudinal extension. The circle lines 212, 212′ are used merely for orientation. The portion 107 is limited by the infeed zone 214 in the region of the end face 10, and by the discharge zone 216 in the region of the base surface 9. The infeed zone 214 and the discharge zone 216 are not part of the sliding region 2 and therefore do not necessarily follow the spindle geometry. The clearance C corresponds to the formula C=(r−r_{P})*2. The K_{G} slides on the sliding surface 2 on the circle line described by the plane 111.

FIG. 6 shows the region of the cranial enlargement of the sliding region 201. The height y_{G} of the cranial enlargement extends between a point of intersection of the normal plane S with the end of the sliding region 2 in the direction of the infeed zone 214 and the point Y_{G}. In this case, the point Y_{G} lies on a straight line K_{G} that intersects L. The straight line K_{G} extends between the point of intersection X_{G} of the normal plane S with the end of the sliding region 2 on the outer surface of the spindle 106 and the point Y_{G}. In this case, the points X_{G} and Y_{G} are arranged on a plane which extends through the end points of the sliding region 2. The two points X_{G} and Y_{G} are mutually spaced. If the cranial elevation is symmetrical, i.e. the ascent and fall are of the same length and each extend over 180°, then the point X_{G} is arranged opposite the point Y_{G}. It is then 180° away from the point Y_{G}. In the case of an embodiment of this kind, a gentle ascent of the cranial elevation can be achieved. If the ascent or the fall of the cranial elevation are steeper, two points X_{G} may be provided. The slope of the cranial elevation begins and ends at these points. Between these two points X_{G}, where no cranial elevation is formed, the implant can be formed so as to be planar and flat, without any elevation or depression. In the preferred embodiment shown, the straight line K_{G} also intersects the center point of the spindle, and Y_{G} lies on the outer surface of the spindle. For the height H_{G} of the sliding region of an implant having a cranial elevation, the following applies: H_{G}′=H_{G}+y. The same relations can be created for the cranial enlargement of the implant, proceeding from the height of the implant.

FIG. 7 shows the region of the cranial lengthening 202 of the implant. The region results between the point Y′ on a straight line K′ and the sectional plane S′. The straight line K′ extends from point X′, which is located on the plane S′ and the outer surface of the spindle 106, to a further point Y′ which is located opposite X′ and represents the maximum of the cranial elevation. In this case, X is located on the opposite side from Y′, i.e. a straight line from X′ to Y′ intersects L. For the height of the implant the following applies: H′=H+x. The region 205 corresponds to the bone contact surface of the outside of the implant in the inserted state. As shown, this region is preferably in parallel with the straight line K′ which shows the maximum dimension of the implant in the region of the base surface. The axis of rotation R of these bone contact surfaces is therefore perpendicular to the straight line K′. An implant of this kind then appears as an implant having a cranial elevation, the inside geometry of which is tilted away from the cranial elevation in the form of a portion of a spindle.

1. Ceramic bone implant for the tribological pairing comprising a spherical sliding partner, the bone implant being formed in a half shell or annular manner and comprising an inner surface which is formed as a sliding region for receiving a spherical sliding partner, wherein the sliding region corresponds to a portion of half a spindle of a spindle torus in the longitudinal extension, the maximum diameter D1 of the sliding region being larger than the diameter of the spherical sliding partner to be inserted, and the minimum diameter D2 of the sliding region being smaller than the diameter of the spherical sliding partner to be inserted, and the radius r of the circle describing the spindle torus, the clearance C, and the radius r_{P} of the sphere of the prosthesis being in the relationship according to Formula I.
C=(r−r_{P})*2 (Formula I).

2. Implant according to claim 1, comprising a first region for introducing the sliding partner and having an end face which represents the transition from the inside to the outer surface in the first region, and a second region which limits the reception of the sliding partner, comprising a base surface which is located opposite the end face, and an outside which comprises an outer surface having a rough and/or structured surface for anchoring the implant in the bone.

3. Implant according to either claim 1, wherein the implant is formed as a half shell and the minimum diameter D2 of the sliding region is 0, or wherein the implant is formed as a half shell and is flattened, and the closed base surface does not touch the spherical sliding partner to be inserted.

4. Implant according to either claim 1, wherein the implant is annular.

5. Implant according to claim 1, wherein the height H_{G} of the sliding region corresponds to 20% of the diameter of the sphere to be inserted, and/or 50-95% of the height H of the implant.

6. Implant according to claim 1, wherein the following applies: 10 μm<C<500 μm.

7. Implant according to claim 1, wherein the implant, preferably the sliding region, is elevated cranially, and/or the implant is annular and the sliding region is enlarged cranially.

8. Implant according to claim 1, wherein the rough surface is a coating.

9. Implant according to claim 1, wherein the rough surface is a porous surface.

10. Implant according to claim 1, wherein the porous surface has a porosity between 50% and 99%, preferably between 60% and 85%, and the pores have a pore size of between 100-1000 μm.

11. Implant according to either claim 7, wherein the porous surface is a porous ceramic.

12. Implant according to claim 9, wherein the porous ceramic is a porous ceramic foam.

13. Implant according to claim 1, wherein the implant is entirely ceramic.

14. Implant according to claim 11, wherein the implant consists of a porous ceramic foam.

15. Use of an implant according to claim 1 in hip, shoulder, elbow, finger joint or toe joint endoprosthetics.
